(19) United States
(12) Patent Application Publication
(10) Pub. No.: US 2024/0074667 A1
(43) Pub. Date: Mar. 7, 2024

(54) SYSTEM

(71) Applicant: ROCKLEY PHOTINICS LIMITED, Cheshire (GB)

(72) Inventors: Kyle RICK, Boulder, CO (US); Roozbeh PARSA, Portola Valley, CA (US); Lok Man CHU, San Marino, CA (US); Vafa JAMALI, Boulder, CO (US); Suresh CHENGALVA, Carmel, IN (US); Kate Leeann BECHTEL, Pleasant Hill, CA (US); Andrew George RICKMAN, Marlborough (GB); Vish KULKARNI, Pasadena, CA (US); Todd NEWHOUSE, Pasadena, CA (US); Jennifer Lynn CORSO, Peoria, AZ (US)

(21) Appl. No.: 18/263,324

(22) PCT Filed: Feb. 3, 2022
(86) PCT No.: PCT/IB2022/000050

§ 371 Date: Jul. 27, 2023

Related U.S. Application Data
(60) Provisional application No. 63/151,521, filed on Feb. 19, 2021.
(60) Provisional application No. 63/146,325, filed on Feb. 5, 2021.

(51) Int. Cl. A61B 5/0205; A61B 5/00; A61B 5/15

(52) U.S. Cl. CPC A61B 5/0205; A61B 5/0022; A61B 5/0024; A61B 5/150801; A61B 5/681; A61B 5/6824; A61B 5/14532; A61B 2560/045; A61B 2560/0462

(57) ABSTRACT

A system comprising: a first module comprising a first sensor, capable of performing biometric sensing at a first location on a patient; and a second module comprising a second sensor, capable of performing biometric sensing at a second location on the patient. The first module comprises a transmitter for transmitting first sensor data, the first sensor data comprising sensing information obtained by the first sensor.

### RELATED APPLICATIONS

The present application claims priority to, and the benefit of, U.S. 63/146,325 filed 5 Feb. 2021 (05/02/2021) and U.S. 63/151,521 filed 19 Feb. 2021 (19/02/2021), the contents of which are incorporated herein by reference in their entirety.

### FIELD OF THE INVENTION

The present invention relates to a system.

### BACKGROUND

In various clinical or home healthcare settings, obtaining biometric data from patients can be advantageous, e.g. to sense levels of chemical compounds (e.g., glucose) in the tissue, to measure other characteristics (e.g., temperature) of the tissue, or to distinguish different kinds of tissue (e.g., to distinguish healthy tissue from diseased tissue).

It is desirous to improve the systems capable of obtaining such data, and the present invention was devised in light of these considerations.

### SUMMARY

Accordingly, in a first aspect, embodiments of the invention provide a system comprising: a first module comprising a first sensor capable of performing biometric sensing at a first location on a patient; and a second module comprising a second sensor, capable of performing biometric sensing at a second location on the patient, wherein the first module comprises a transmitter for transmitting first sensor data, the first sensor data comprising sensing information obtained by the first sensor.

By having a first and second module which can perform biometric sensing at different locations on the patient, a number of advantages may be provided. For example, redundant measurements can be taken. Further, differential measurements of a same biomarker can be taken to enhance the fidelity of the signal (e.g. lower the signal-to-noise ratio), especially when calibrating with a known source. It can also provide insight into anatomical differences.

The system may have any one or, to the extent that they are compatible, any combination of the following optional features.

The first and/or second modules may form discreet sub-components of a larger device or monitoring system. That is, the system may be a single device (including the first and second modules) or may be a monitoring system where the modules are provided in distinct devices. For example, the first and second modules may be discrete integrated circuits (electronic, photonic, or a mixture thereof) which are located within a patient monitoring system or device. In some examples the first and second modules are provided in separate devices which are independently positionable on the patient. For example, the first module may be located in one element worn or attached to the patient at a first location (e.g. in a wristband worn by the patient) whereas the second module may be located in another element worn or attached to the patient at a second location different to the first location (for example, an article of clothing, a chest strap, an earring, an ankle strap, an adhesive patch, an arm or bicep strap, or a ring).

The system may include further modules including sensors capable of performing biometric sensing at further locations on the patient. The system may form a distributed sensor network. The data from the sensors can be provided to a processing unit, which may be in one of the first or second modules or may be a separate module. The processing unit may be, or may be in, a third module or other module, for example a mobile device (e.g. cell phone, tablet, laptop, or other computer) which is in one- or two-way communication with the modules containing sensors. The processing unit may be a remote unit, for example in a cloud computing system connected to the modules via a network connection.

One of the modules may be deemed a main unit or main module and may control the other modules of the system. For example, it may prompt them to switch on or off and to take readings or perform calibration routines.

The modules may each include a plurality of sensors, and/or each sensor may be configured to undertake more than one type of biometric sensing. In either case, a wealth of biometric information is available allowing for multidimensional analysis across several factors.

One or more of the modules may be, or include, an optical sensing module or optical sensor. The or each optical sensing module or optical sensor may include a transmitter photonic integrated circuit, comprising a plurality of lasers where each laser of the plurality of lasers operates at a different wavelength to the others. The optical sensing module may include an optical manipulation region which can include one or more of: an optical modulator, an optical multiplexer, and additional optical manipulation elements. The module may include one or more optical outputs for light originating from the lasers. The optical sensing module may be, for example, as disclosed in WO 2021/116766 A1 the disclosure of which is incorporated by reference in its entirety.

In some examples, one or more of the sensors may be a photoplethysmograph, speckleplethysmograph (SPG), or a spectrophotometer operating at one or more wavelength bands such as visible, near-infrared or short-wave infrared.

In one example, the optical sensors are provided on a top and bottom of a wristband (i.e. on a dorsal and ulnar/radial portion of the wristband) with electronic components disposed across the band. Positioning the sensors over the ulnar/radial portion of the patient's hand enhance the data obtained by the sensors, for example because of the increased vascularization in that location as compared to (for example) the dorsal region of the patient's hand.

The system may further comprise a third module comprising a receiver, the third module being configured to receive the first sensor data and to combine the first sensor data with other sensor data. The third module may be the second module. The other sensor data may comprise sensing information obtained by the second sensor. The first module may comprise a receiver for receiving a signal from the second module.

The third module may be a mobile phone. The first module may comprise a receiver for receiving a signal from the third module. The other sensor data may comprise sensing information obtained by the second sensor.

The transmitted may be a wireless transmitter. The transmitter may be a Bluetooth (RTM) transmitter or a WiFi (RTM) transmitter. The transmitter may be a near-field communication (NFC) transmitter.

The first sensor and/or the second sensor may be capable of performing biometric sensing of a type selected from the group consisting of blood sugar measurements, blood glucose measurements, core body temperature measurements, hydration level measurements, blood pressure measurements, breathing rate measurements, SpO₂ measurements, heart rate measurements, heart rate variability measurements and combinations thereof.

The first module may not include a receiver. In other words, the first module may be arranged to communicate in a one-way fashion with the second or third module by transmitting to them but being unable to receive information from them. In some examples the second module does not include a receiver.

The first sensor and the second sensor may be configured to perform redundant measurements. That is, the first and second sensor may be configured to perform biometric sensing of a same type.

The first and second modules may be located within a wearable device. In this sense, the system may be considered a wearable device. The wearable device may be or include a wristband, and the first and second modules may be located at different circumferential positions around the wristband (i.e. at different points around a circumference of the wristband, the wristband having a generally circular or ovoidal shape or surface). The wristband may include a clasp or other fixing mechanism which can be coupled or uncoupled so as to form a complete or split band respectively. The clasp or other fixing mechanism may be used to form electrical connections between components in or on the wristband.

The system may further include an indicator module, configured to provide feedback to a user as to the position of the wearable medical device on the user. The indicator module may be, for example, a plurality of LEDs which illuminate to indicate the wearable medical device is positioned at predetermined location on the user. The system may further include an adjustment mechanism to vary a dimension of the medical device. The wearable medical device may be a wristband, including, or being connectable to, a watch module configured to display the time, the wristband being arranged such that the watch module is or would be disposed on a dorsal portion of a wristband and the first and second modules are disposed on a radial or ulnar portion of the wristband. By dorsal portion, it is typically meant the upper portion of the wrist on the same side of the hand to the fingernails. Whereas the radial or ulnar portions are located circumferentially around the wrist, on a lower portion e.g. on the same side as the palm of the hand. The wearable device may include a flexible battery unit.

The system may further include a notification module, configured to feedback to a user a value of a biomarker derived from the sensor data. For example, the notification module may be provide haptic or visual feedback (e.g. through a vibrator or LED) that a value of a biomarker is outside of a predetermined range or exceeding a predetermined threshold.

The watch module may include one or more transmitters for connecting and communicating with another device, such as a mobile phone.

The wristband may be formed of a flexible material (e.g. rubber), or formed of a plurality of links of a relatively inflexible material (but so as to be conformed as a device to a shape of the patient by virtue of the movable links). The wristband may be, for example, about 3 mm in thickness and may be about 20 mm wide.

The first sensor and the second sensor may be configured to perform biometric sensing of a same type on different locations of the patient. One or both of the first module and the second module may be configured to derive a biomarker value from sensor data from both the first sensor and the second sensor. In some examples, a differential measurement is taken which may include determining a difference between the biomarker value as derived from the sensor data of the first sensor and the biomarker value as derived from the sensor data of the second sensor. For example, the system may determine a first blood pressure measurement at a point proximal to the heart of the patient using the first sensor and a second blood pressure measurement from a point distal to the heart of the patient using the second sensor. The system may be able to determine, therefore, from a difference between the first and second blood pressure measurements that the patient may have poor blood circulation or perfusion.

One or both of the first module and the second module may be configured to receive sensor data from both the first sensor and the second sensor, and to derive a biomarker value from the sensor data from only one first and second sensor. The relevant module may examine the sensor data from the first sensor and the second sensor and chose to derive the biomarker value form the sensor data with the highest quality.

### BRIEF DESCRIPTION OF THE DRAWINGS

Embodiments of the invention will now be described by way of example with reference to the accompanying drawings in which:

FIG. 1 shows a system;

FIG. 2 shows a variant system;

FIG. 3 shows a wearable device; and

FIG. 4 shows a variant wearable device.

### DETAILED DESCRIPTION AND FURTHER OPTIONAL FEATURES

Aspects and embodiments of the present invention will now be discussed with reference to the accompanying figures. Further aspects and embodiments will be apparent to those skilled in the art. All documents mentioned in this text are incorporated herein by reference

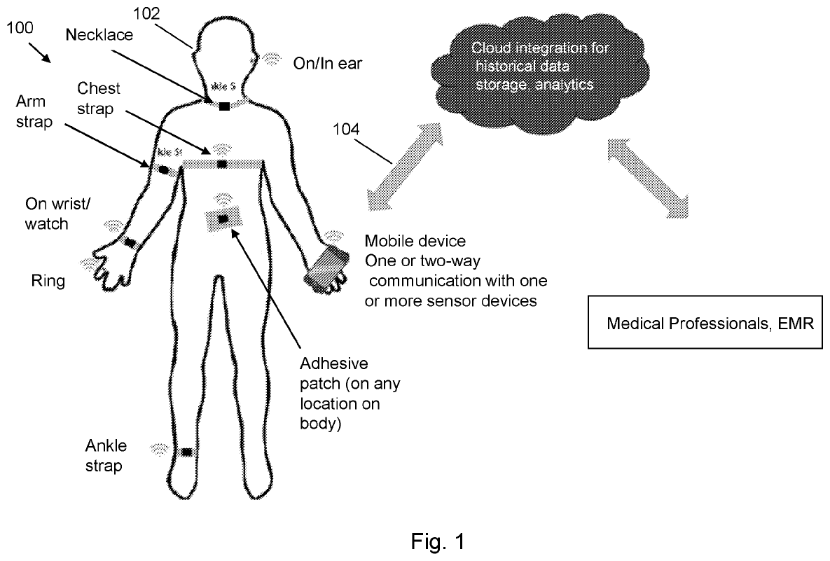

FIG. 1 shows a system 100. The system includes a first module comprising a first sensor, capable of performing a biometric sensing at a first location on a patient. In the system shown in FIG. 1, the first module is one of: a necklace; a chest strap; an earring; a device worn in the inner-ear; a wristband/watch; a ring; an ankle strap; an adhesive patch located on the body; an arm or bicep strap; or a mobile device. The second module is another of a chest strap; an earring; a wristband/watch; a ring; an ankle strap; an adhesive patch located on the body; an arm or bicep strap; or a mobile device. For example, the first module may be a wrist strap and the second module may be a chest strap. The first module includes a transmitter for transmitting first sensor data, where the first sensor data includes sensing information obtained by the first sensor.

The sensors in the first module and second module, as described above, are configured to (and so capable of) performing biometric sensing at their respective locations on the patient. The sensors can be configured to sense: blood sugar level; blood glucose level; core body temperature; hydration level; blood pressure; breathing rate; SpO₂ level; heart rate; heart rate variability, and combinations thereof. The sensors may do so by including photonic components, for example in a small form factor photonic integrated circuit (PIC), as combined with an application specific integrated circuit and/or flexible electronic substrate. The PIC can include, for example, two or three lasers with different wavelengths in the red and near infrared ranges by combining SPG and PPG data.

Where a mobile device forms a part of the system, it can be in one- or two-way communication with the other module(s) including the other sensor(s). For example, the module(s) including the other sensor(s) may be configured to transmit only to the mobile device and may not be able to receive signals from the mobile device.

The mobile device can process the received sensor data to derive biometric markers (e.g. heart rate, glucose level, etc.). Additionally or alternatively, the sensor data can be transmitted to a cloud computing system for cloud integration facilitating historical data storage and more powerful analytical techniques than might be executable on a mobile device. The data can also be shared with medical professionals, either directly or through use of an electronic medical records (EMR) system. This can be implemented either via the cloud (as shown) or directly from the mobile device.

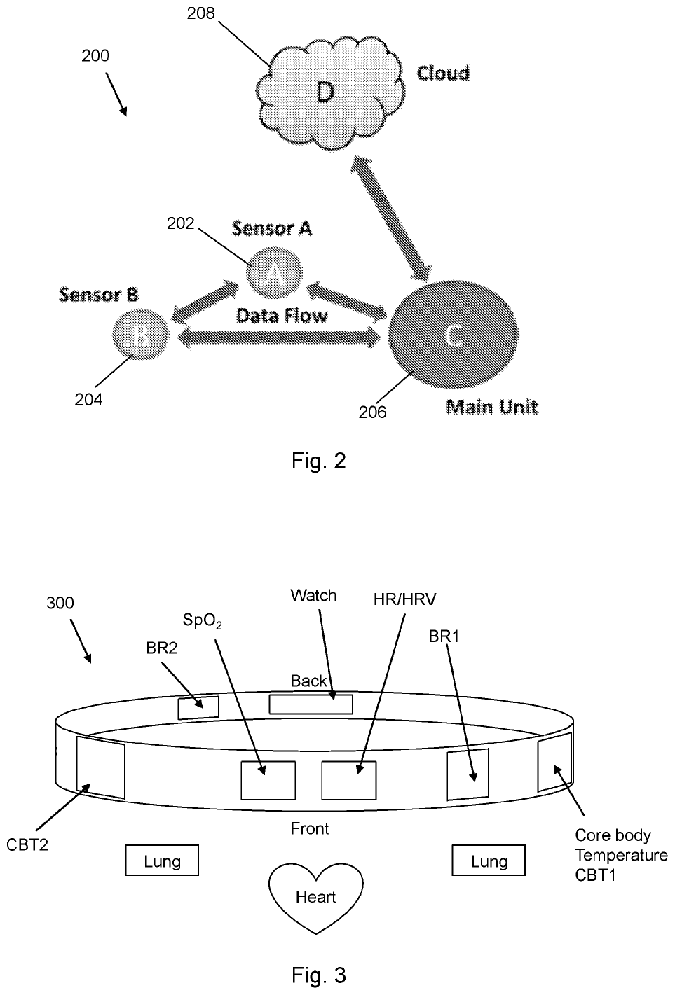

FIG. 2 shows a schematic diagram of a system 200 including three modules. A first module 202 includes sensor A which is configured to perform biometric sensing. A second module 204 includes sensor B which is also configured to perform biometric sensing. A third module 206, termed the main unit, is in communication with the first module 202 and second module 204 (which are also in communication with one another). The third module may contain a sensor configured to perform biometric sensing, or may not and may function instead as a processing and/or communication unit only. In this example the third module 206 is connected to a cloud computing system 208 of the type discussed previously, which may perform further analytical techniques or storage of the data obtained from the sensors.

In some examples, the sensors in each module are configured to sense different kinds of biometric data (for example from the list discussed above) or they may be configured to sense the same kind of biometric data, or subsets of the sensors may be configured to sense the same kind of biometric data whilst yet other sensors or subsets of sensors are configured to sense different kinds of biometric data. Each module may include more than one sensor and so the different sensors within a module may respectively sense different kinds of biometric data.

In examples where the sensors of respective modules are configured to sense the same biometric data, the main unit may decide which data to use between the data from the different sensors based on a quality score or other ranking. The main unit may also be configured to utilise the data from both (or all) sensors in the derivation of a given biometric marker. This can be done, for example, through use of differential analysis to provide enhanced accuracy for a given biomarker or to investigate the change in a biomarker as a function of anatomical position.

Additionally or alternatively the sensors may perform redundant measurements (i.e. both recording the same kind of data in case one or the other fails during a given data acquisition run).

FIG. 3 shows a wearable device 300. In this example, the wearable device 300 is a wristband suitable to be worn around the wrist of a patient. The wristband includes a number of sensors, for example breathing rate sensor 1 (BR1), breathing rate sensor 2 (BR2), core body temperature 1 sensor (CB1), core body temperature 2 sensor (CB2), saturation percentage of oxygen sensor (SpO₂), and heart rate/heart rate variability sensor (HR/HRV).

In this example the wearable device includes a watch module which is configured to display the time. The watch module is located on a back side of the wearable device, which corresponds to the dorsal region of the wristband and so the dorsal portion of the patient when the wristband is on the patient. Whereas at least some of the sensors (which may be included in their own modules) are located on a radial or ulnar portion of the wristband to enhance data collection. The watch module, in this example, includes a transmitter (e.g. a Bluetooth (RTM) transmitter) for transmitting the data from the sensors.

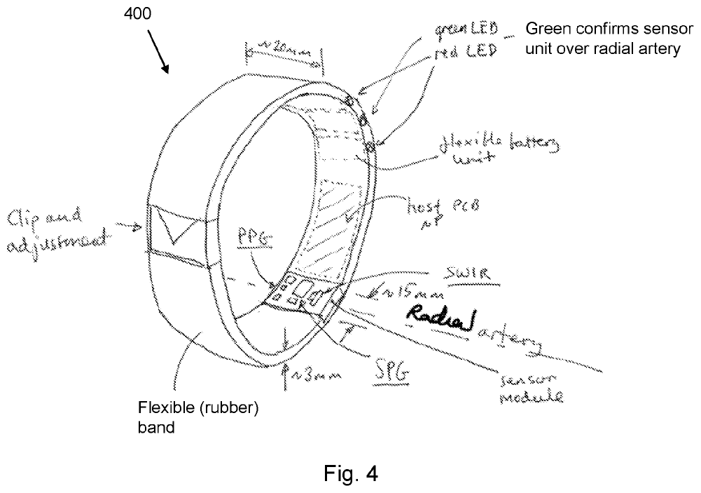

FIG. 4 shows a variant wearable device 400. This wearable device 400 is also a wristband, which is about 20 mm wide (i.e. as measured in a direction aligned with the arm of the patient when worn) and is about 3 mm thick (as measured in a radial direction of the wristband). The wristband 400 includes a clip and adjustment mechanism, so that the ring formed by the wristband can be broken or closed by use of the clip or clasp, and the radius or circumference of the wristband can be varied through use of the adjustment mechanism. In some examples the clip or clasp includes electrical connectors which connect components of the wristband when closed.

The wristband 400 in this example is formed from a flexible material, such as rubber, so as to conform to the shape of the patient's wrist. In other examples, not shown, the wristband is formed of a plurality relatively inflexible links or elements which are connected together so as to be respectively pivotable. In this manner, the wristband can still conform to the shape of the patient's wrist whilst not being formed of a flexible material.

The wristband 400 in this example includes an indicator unit to notify the user if they have placed the wristband in a correct predetermined position on their person. For example, the wristband 400 in this example includes a module which includes a PPG sensor, SWIR sensor, and SPG sensor. The indicator unit notifies the user if these sensors have been positioned above the patient's radial artery, for example by illuminating one or more red LEDs (light emitting diodes) when the unit is incorrectly positioned and illuminating one or more green LEDs when the unit is correctly positioned. The wristband 400 includes a flexible battery unit, as well as a host PCB which can provide the third module. The sensor module in this example includes the SWIR sensor, SPG sensor, and PPG sensor.

The features disclosed in the description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

All references referred to above are hereby incorporated by reference.

1. A system comprising:
a first module comprising a first sensor, capable of performing biometric sensing at a first location on a patient; and
a second module comprising a second sensor, capable of performing biometric sensing at a second location on the patient,
wherein the first module comprises a transmitter for transmitting first sensor data, the first sensor data comprising sensing information obtained by the first sensor.

2. The system of claim 1, comprising:
a third module comprising a receiver, the third module being configured to receive the first sensor data and to combine the first sensor data with other sensor data.

3. The system of claim 2, wherein the third module is the second module, and wherein the other sensor data comprises sensing information obtained by the second sensor.

4. The system of claim 3 wherein the first module comprises a receiver for receiving a signal from the second module.

5. The system of claim 2, wherein the third module is a mobile telephone.

6. The system of claim 5, wherein the first module comprises a receiver for receiving a signal from the third module.

7. The system of claim 5, wherein the other sensor data comprises sensing information obtained by the second sensor.

8. The system of claim 1, wherein the transmitter is a wireless transmitter.

9. The system of claim 8, wherein the transmitter is a Bluetooth transmitter.

10. The system of claim 8, wherein the transmitter is a WiFi transmitter.

11. The system of claim 8, wherein the transmitter is a near-field communication transmitter.

12. The system of claim 1, wherein the first sensor and/or the second sensor is capable of performing biometric sensing of a type selected from the group consisting of blood sugar measurements, blood glucose measurements, core body temperature measurements, hydration level measurements, blood pressure measurements, heart rate and heart rate variability, SpO2, and combinations thereof.

13. The system of claim 1, wherein the first module does not include a receiver.

14. The system of claim 1, wherein the first sensor and the second sensor are configured to perform redundant measurements.

15. The system of claim 1, wherein the first and second modules are located within a wearable device.

16. The system of claim 15, wherein the wearable device includes a wristband, and the first and second modules are located at different circumferential positions around the wristband.

17. The system of claim 15, further including an indicator module, configured to provide feedback to a user as to the position of the wearable device on the user.

18. The system of claim 15, further including an adjustment mechanism to vary a dimension of the wearable medical device.

19. The system of claim 15, wherein the wearable medical device is a wristband, including, or being connectable to, a watch module configured to display the time, the wristband being arranged such that the watch module is or would be disposed on a dorsal portion of a wristband and the first and second modules are disposed on a radial or ulnar portion of the wristband.

20. The system of claim 1, wherein the first sensor and the second sensor are configured to perform biometric sensing of a same type on different locations of the patient.

21. The system of claim 20, wherein one or both of the first module and the second module is configured to derive a biomarker value from sensor data from both the first sensor and the second sensor.

22. The system of claim 20, wherein one or both of the first module and the second module is configured to receive sensor data from both the first sensor and the second sensor, and to derive a biomarker value from the sensor data from only one first and second sensor.
